(19) United States
(12) Patent Application Publication
(10) Pub. No.: US 2009/0209918 A1
(43) Pub. Date: Aug. 20, 2009

(54) METHOD AND DEVICE FOR DIALYSIS

(75) Inventor: Rick Berglund, San Diego, CA (US)

Correspondence Address: MAYER & WILLIAMS PC, 251 NORTH AVENUE WEST, 2ND FLOOR, WESTFIELD, NJ, 07090, US

(73) Assignee: IMTEC, LLC, San Diego, CA (US)

(21) Appl. No.: 12/206,674

(22) Filed: Sep. 8, 2008

Related U.S. Application Data
(60) Provisional application No. 60/967,775, filed on Sep. 7, 2007.

(51) Int. Cl. A61M 5/32; A61M 25/00

(52) U.S. Cl. 604/175; 604/264

(57) ABSTRACT

A method and system are provided for performing dialysis. The system provides needleless vascular access which reduces the repeated traumas of conventional dialysis by providing a vascular graft and port combination which interfaces with a specially-designed catheter system. The installed implant is low profile, small and stable, and saddles the vessel to secure the protruding surface tissue above. The stability of the port is maintained by features that encompass the vessel and the surrounding tissue. These features are enhanced by the catheter attachment, which also provides reduced trauma and provides convenient access to the implant for performance of dialysis functions.

### CROSS-REFERENCE TO RELATED APPLICATIONS

This application claims the benefit of U.S. Provisional Patent Application Ser. No. 60/967,775, filed Sep. 7, 2008, entitled “Method and Device For Dialysis”, owned by the assignee of the present invention and herein incorporated by reference in its entirety.

### FIELD OF THE INVENTION

The invention relates to dialysis, and more particularly to methods and devices for accessing the bloodstream to conduct dialysis.

### BACKGROUND

It is estimated that 1.2 million people worldwide suffer from end stage renal disease. The US has approximately 375,000 such patients and the number is growing at a rate of about 7% every year. Thus, the total patient population is expected to double in ten years. Also currently, 3 million people with kidney failure go undiagnosed or untreated, particularly in developing countries.

Currently, more than 65,000 deaths occur every year as a result of kidney failure. Over the last five years, the number of new patients with kidney failure has averaged more than 90,000 annually. The total current cost of treating kidney failure in the US is approximately $17.9 billion.

Dialysis is a renal replacement therapy that provides an artificial replacement for lost kidney function. Rather than being a cure for a kidney disease, it is a life support treatment. Dialysis requires the removal of blood from the patient, treatment of the blood, and then replacement of the treated blood back into the patient. The treatment of the blood mainly involves removal of undesired solutes via passing the blood on one side of a semipermeable membrane.

There are approximately 3,600 dialysis facilities and 225 transplant facilities in the US, of which only about 260 dialysis clinics are hospital-based. The success of the treatments provided by these centers is mixed. Prior dialysis treatments are associated with high complication rates, including trauma and damage to blood vessels, scarring, minor and major bleeding, clotted vessels, extremity vascular damage (such as fistulas or grafting), localized infections, aneurysms of grafts due to multiple “sticks” or needle punctures, high venous or arterial pressures due to stenosis, aneurysms, and mental stress to the patient.

In more detail, years spent on dialysis using conventional needles creates access location problems for the patient, and forces surgeons to find new placement locations. In addition, the scarring left from needle punctures after years of “sticks” is visually unappealing, and often creates self-esteem problems for patients. This may be compounded with current grafts, which can bulge and protrude half an inch or more.

Even a skilled dialysis technician can encounter difficulties placing needles into grafts, and these difficulties can include causing a hole or tearing other vessels, which can lead to internal bleeding. Needles can also move to the side of the vessel after placement, slowing blood flows, which can lower blood clearances and increase dialysis time. The same can potentially even clot the system.

Site healing can be slow due to patient conditions. For example, repeated punctures in and around the same site can cause the buildup of scar tissue and vessel leakage, which can create a fear of vessel bleeding at any time. The patient blood pressure can also affect a graft life span.

### SUMMARY

Embodiments of the invention provide a method and system for avoiding the problems of the prior art. The system provides needleless vascular access which reduces the repeated traumas mentioned above by providing a vascular graft and port combination which interfaces with a specially-designed catheter system.

A vascular port, of a small size, implanted into an extremity of the body has many benefits for a long duration of dialysis, and would be highly desired by dialysis patients, who could be essentially assured of the success of their next procedure.

The implant port may be significantly more than just a passage to the bloodstream. The device is designed to encompass and address the issues that create problems. The implant is low profile, small and stable, and saddles the vessel to secure the protruding surface tissue above. The stability of the port is maintained by features that encompass the vessel and the surrounding tissue. These features are enhanced by the catheter attachment, which also provides reduced trauma.

The graft is used for the blood flow to the implant. The PTFE graft is not punctured, and the graft length is long enough to attach to and from the arterial and venous system. This provides for a shorter segment of graft material and lessens the clotting potential of foreign material that is recognized by the body. The smooth internal saddle-shaped flange also lessens flow turbulence.

Topical applications may be employed to protect the exit site from contamination, and silver or oxygen protection may be used. The external port is low profile, which is more comfortable to the patient, is less unsightly, and has less chance for manipulation which can lead to irritation and infection.

Catheter use may be automated in some implementations, and this ensures accurate placement of seals, etc., leading to lesser phlebotomy skills needed for catheterization. The catheter essentially takes control of most operations needed for interfacing.

Advantages may include one or more of the following.

The catheter system provides a low-bulk and low-complexity device. Operations are automated to various degrees, including automated seal extraction and insertion. The system is low cost and can ease clinical operations. The system is self-locking and sealing. The system is easily flushed. The system may be self-aligning. The system may be operated from pressurized saline. The system can adapt to a module dialyzer, such as for home care. The system may employ dial-type rotation controls, and the same may be automated for motor operation and programming. The system is single-use disposable, and minimal technical training is required. The tip of the catheter may be protectable by a cover. The system can operate at low pressures.

The implant system also provides a low-bulk and low-complexity device. No replaceable parts need be provided, and no valving need be present. Only a narrow protrusion through the skin is needed. The system enjoys low to no infection potential. Easy access is allowed, and the system is designed to be at least vertically and rotationally stable. The implant has an automated seal, and causes low turbulence to the bloodstream. Little manipulation is required, and thus there no little trauma to the exit site. No needle punctures are required, and the system enjoys positive pressure sealing.

Additional advantages will be apparent from the description that follows, including the figures and claims.

### BRIEF DESCRIPTION OF THE DRAWINGS

FIG. 1 shows an external view of the implant, connected in a forearm location.

FIG. 2 shows an external view of the catheter interfaced to the implant for dialysis. The forearm location provides ease for the control dial.

FIG. 3 shows a perspective view of the catheter and implant in an interfaced configuration.

FIG. 4 shows an extended catheter tip for the priming mode of the first embodiment.

FIG. 5 shows a side interface view of the first embodiment of the catheter device and the implant.

FIG. 6 shows the catheter according to the first embodiment and the implant together, where the blood catheter is retracted for the interfacing procedure.

FIG. 7 shows a sectional view of a first embodiment of the catheter showing the blood catheter extended through the implant's passageway.

FIG. 8 shows blood flow to and from the implant.

FIG. 9 shows an end-on view of the implant.

FIG. 10 shows a side view of a first embodiment of the locking interface, on the catheter portion.

FIGS. 11 and 12 show perspective and side views, respectively, of a first embodiment of an interface locking mechanism between the catheter and the implant, illustrating in particular a side view of the catheter engaging the implant (FIG. 12) and a perspective view of a portion of the catheter locking mechanism (FIG. 11).

FIG. 13 shows locking edges for use in the implant of the embodiment of FIG. 12.

FIGS. 14 and 15 show stem seal locking tabs and a sealing surface which form part of the embodiment of FIG. 12 for an interface mechanism between the catheter and the implant.

FIG. 16 shows a retracted tab in the embodiment of FIG. 12.

FIG. 17 show side views of unlocked and locked configurations of the catheter and implant in a second embodiment of an interface locking mechanism between the catheter and the implant.

FIG. 18 show top views of unlocked and locked configurations of the catheter and implant.

FIG. 19 shows a more detailed view of the locking interface.

FIG. 20(A) shows a side view of the implant.

FIG. 20(B) shows an exploded view of the implant, showing the disposition of the stem seal locking tabs relative to the remainder of the construction.

FIG. 21 shows the external blood circuit.

FIG. 22 shows a top schematic view of a first embodiment of the catheter device according to the invention.

FIG. 23 shows a schematic view of the first embodiment of the tube bundle assembly.

FIG. 24 shows an end-on view of the first embodiment of the tube bundle assembly.

FIG. 25 shows a left-side view of the first embodiment of the catheter device.

FIG. 26 shows a right-side view of the first embodiment of the catheter device.

FIG. 27 shows a schematic view of the tube bundle cylinders.

FIG. 28 shows a schematic view of the port configuration of the first embodiment, particularly referring to the tube bundle assembly, and in particular in a priming mode.

FIG. 29 shows a schematic view of the tube bundle of the first embodiment, in the plug or stem or stem seal retraction (as well as catheter infusion) mode.

FIG. 30 shows a schematic view of the tube bundle according to the first embodiment, in the catheter retraction mode.

FIG. 31 shows a schematic view of the tube bundle according to the first embodiment, in the plug infusion mode.

FIG. 32 shows another view schematic view of the tube bundle according to the first embodiment.

FIG. 33 shows a side sectional view of a porting unit which may be employed in the second embodiment of a catheter of the invention.

FIG. 34 shows a side sectional view of another embodiment of a porting unit which may be employed in the second embodiment of a catheter of the invention.

FIG. 35 shows additional details of the porting unit of FIG. 34.

FIG. 36 shows a logic diagram for the porting unit cam positions, indicating which lines are occluded or not, for various stages of the procedure.

FIG. 37 shows a vent dial which may be employed with the second embodiment of the catheter.

FIG. 38 shows another embodiment of a vent dial which may be employed with the second embodiment of the catheter.

FIG. 39 shows a schematic view of the first embodiment, including a cam driving disk.

FIG. 40 shows a view of the rotation dial, according to the first embodiment, which is disposed on one end of the catheter device.

FIG. 41 shows a more detailed view of the stem seal retraction.

FIG. 42 shows a more detailed view of the stem seal insertion.

FIG. 43 shows a more detailed view of the stem seal.

FIG. 44 shows a schematic view of a home dialysis machine, with the module dialyzer installed.

FIG. 45 shows a sectional view of the home dialysis machine.

FIG. 46 shows a top sectional view of the module dialyzer.

FIG. 47 shows a flowchart illustrating a method for conducting dialysis.

### DETAILED DESCRIPTION

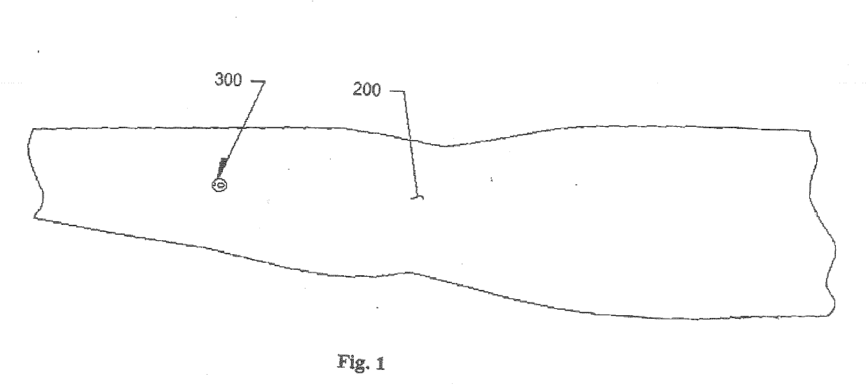

FIG. 1 shows an external view of an installed implant, connected in a forearm skin 200 location, without a catheter interfaced. As can be seen, the exposed portion is minimal, enhancing patient acceptance. Of course, depending on patient requirements, the size may vary as is appropriate.

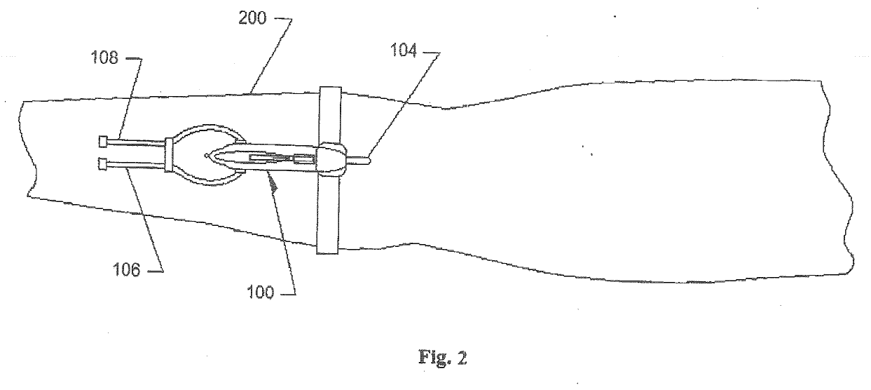

FIG. 2 shows an external view of the catheter 100 as the same is interfaced to the implant 300 at a location on the skin 200 for dialysis. The forearm location provides ease of access for a control dial 104. This figure also shows an arterial supply tube 106 and a venous return tube 108. It should be noted that while the control dial 104 is shown as occupying a plane substantially perpendicular to the axis of the catheter, the same may be conveniently disposed at various angles, substantially facing upward towards the user, e.g., at a 45° angle. This may provide in certain embodiments a convenient angle for user operation.

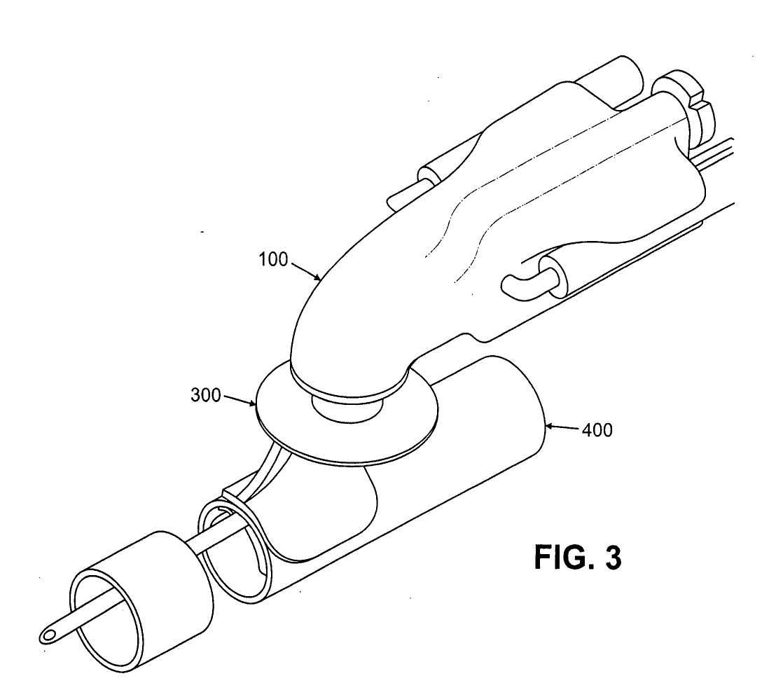

FIG. 3 shows a perspective view of the catheter and implant in an interfaced configuration, showing a catheter 100 and an implant 300, the implant 300 disposed on a graft 400.

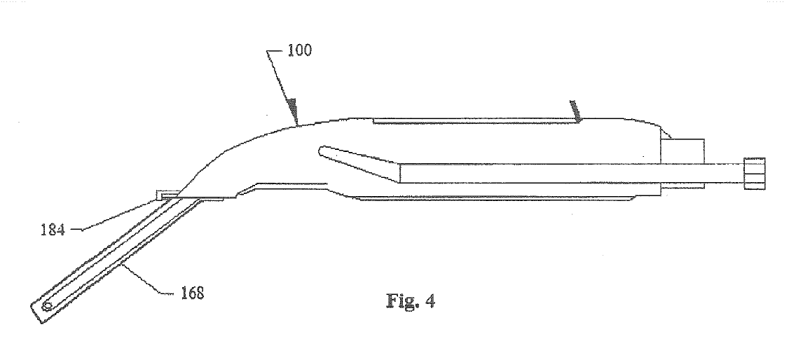

FIG. 4 shows a side cross-sectional view of the catheter 100 as the same may be provided for use. A protective cap 168 is employed to reduce ingress of deleterious elements into the catheter when the catheter is packed, shipped, and stored prior to use. The protective cap 168 also plays a role in the priming procedure in certain implementations as will be described. In use, the cap 168 is removed and the catheter 100 is attached to the implant. Locking mechanisms of various configurations may be employed, certain of which are described below.

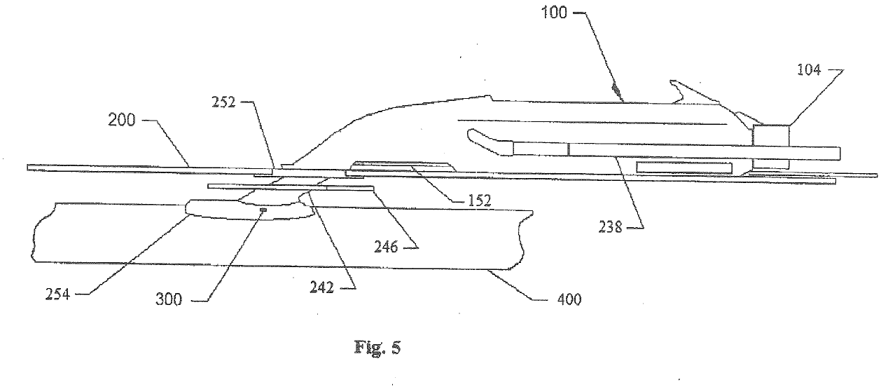

FIG. 5 shows a side interface view of the catheter device 100 and the implant 300, showing the two in a locked configuration around the patient's skin 200. The implant 300 includes an external interface surface 252, a neck 242, and a saddle 254 which mates the implant to a graft. A stabilizing flange 246 is employed which gives the implant rotative and vertical stability, as well as a platform for ingrowth.

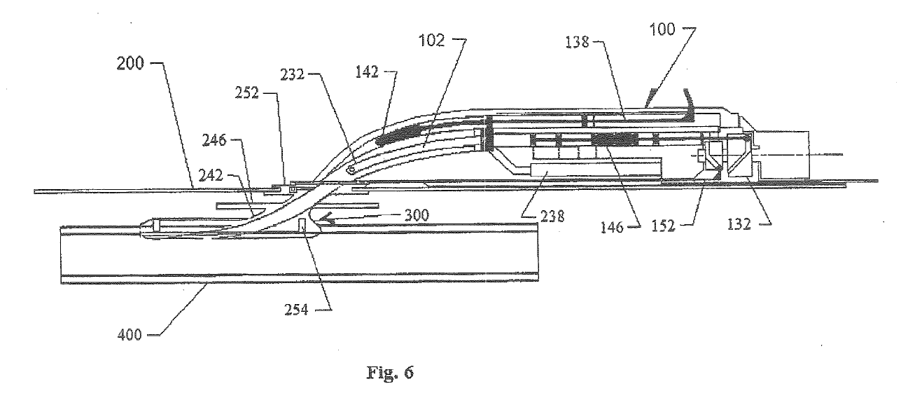

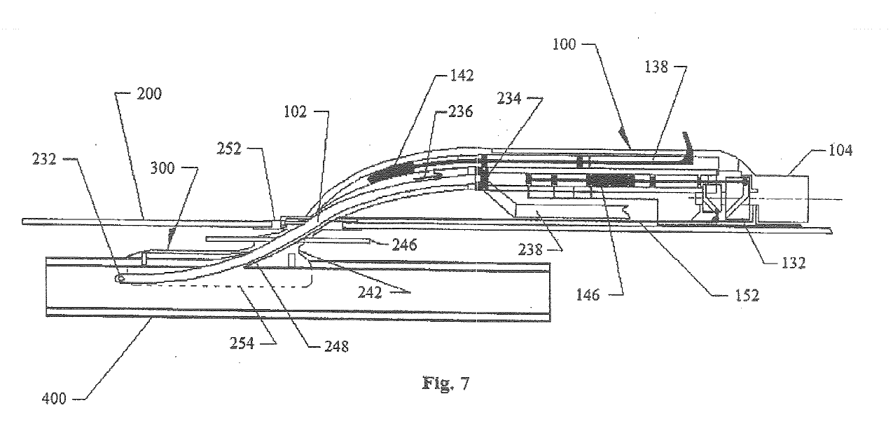

FIG. 6 shows the catheter and the implant together, where the blood catheter 102 is retracted for the interfacing procedure. In the figure, the implant 300 is coupled to a graft 400. FIG. 7 shows the system of FIG. 6, but where the blood catheter is extended for a dialysis procedure.

FIG. 6 also shows a blood catheter 102 extended through the implant's passageway, the blood catheter being protected by the cap 168 when not in use. The blood catheter 102 has a distal tip that returns the dialyzed blood into the venous circuit. The blood catheter 102 has a proximal tip 234 which seats inside the catheter structure and that allows the porting of the venous return. The inflow or arterial blood travels upward on the outside of catheter 102 (lumen 236). Lumen 238 is connected to the venous return. A cam forces a locking rod 152 forward when going from the unlocked to the locked position, forcing a locking post into a receiver (described below). Various other components are shown as will also be described.

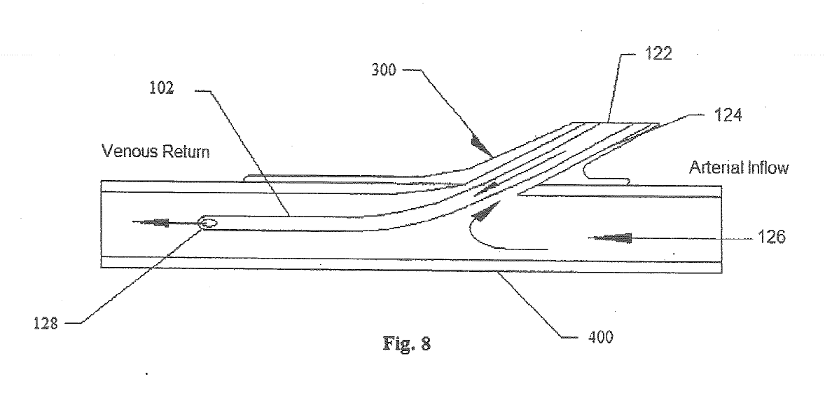

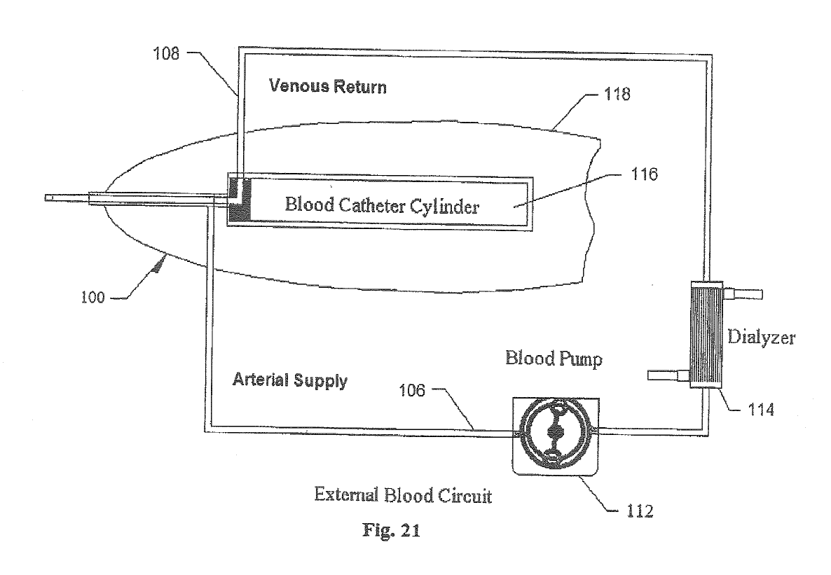

The venous return from the dialysis machine may be disposed, e.g., 1.5 to 2.0 inches from the arterial inflow to the dialysis machine. This may be seen more clearly by reference to FIG. 8, which shows blood flow to and from a dialysis machine from the implant. In particular, blood flows in the patient's vessel in the direction indicated by arrow 126. A blood pump 112 (see FIG. 21) draws blood into the arterial supply tube 106 and more particularly into an exterior concentric blood lumen 124. Following dialysis, blood is returned via a central blood lumen 122. The point at which blood is returned to the vessel, i.e., a distal tip 128 of the blood catheter, is disposed downstream of the point at which blood is removed from the vessel. This reduces mixing and re-dialyzing of blood.

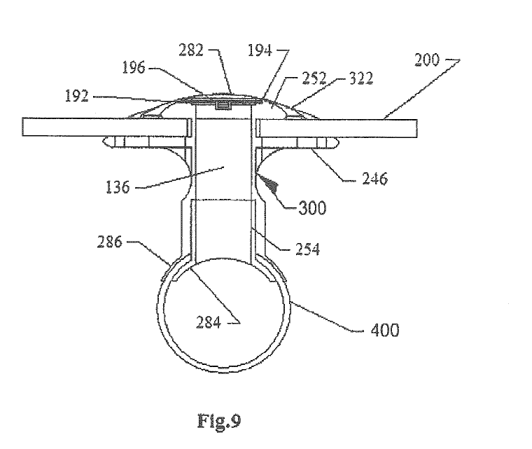

Referring to the implant, FIG. 9 shows an end-on view of the implant 300, indicating how the same penetrates the surface skin and tissue layer 200 and attaches to a graft 400 below. A protective seal 282 is shown, along with the stability flange 252. A post receiver 196 is shown, along with locking tabs 192 and 194, which are employed for connecting to the catheter 100 (described below). The connection to the graft includes an internal lumen saddle 284 and an external lumen saddle 286 configured on the distal end of the implant structure. The two are press-fit around a circular punched hole in the graft until a tight structure is obtained. The punching is advantageous because it allows the cylindrical portion of the internal lumen saddle to fit through the hole and then interface with the external mating surface of the implant's external saddle.

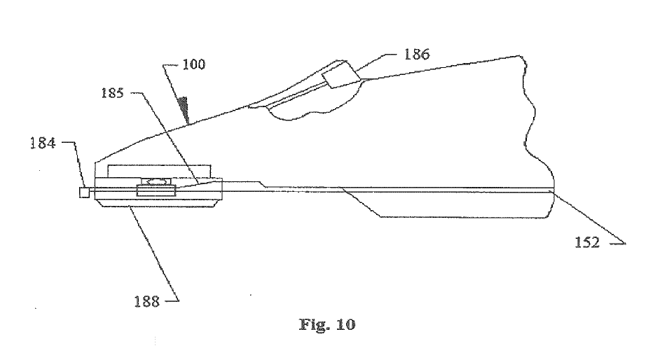

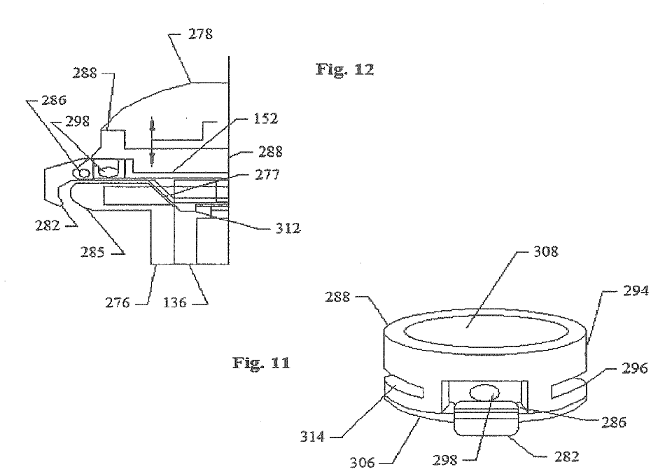

FIG. 10 shows a side view of a first implementation of the locking interface as well as the forward portion of the housing of the catheter, showing details of the locking interface mechanism, which is described in greater detail below. FIGS. 11 and 12 show additional details of the locking mechanism, with certain details disposed thereon, including rocker arms, related cams, and a compression seat on the implant's sealing surface area 285.

In particular, FIG. 10 shows the catheter 100 which may include a hyper injection port 186 at a portion thereof. The locking rod 152 runs at least a portion of the length of the catheter, and terminates at a post (for the seal door) 184, which in FIG. 10 is downwardly-depending from a distal extremity of the locking rod 152. The operation of the locking rod 152 is described elsewhere. The motion of the locking rocker arms causes an upper portion of the implant to be forced upward against a sealing surface 188 on the catheter, the same being forced downward against the implant. In particular, surfaces 277 and 306 (see FIG. 12) are forced together, sealing the interface.

FIGS. 11 and 12 show additional details of the locking and compression mechanism that may be employed to attach the catheter 100 to the implant 300. As shown in FIG. 12, as a cam moves forward and upward, the rocking arm is attached to an outer rim, sealing the two surfaces. In particular, FIG. 12 is a sectional view of the distal end 278 of the catheter and implant interface showing the rocker arm position. As locking rod 152 displaces forward, the ramp 185 displaces upward a contact head 298 of the rocker arm to a maximum ramp position, forcing the contact surface 282 to pivot about hinge 288 to tightly contact surface 282 to surface 285 for compression of seating surfaces 306 to 277. FIG. 11 shows a detail of the housing 288. A first annulus 296 is spaced from a second annulus 294 to form a space 314 through which the locking rod 152 passes. Space 308 allows entry of the catheter and the stem seal 136.

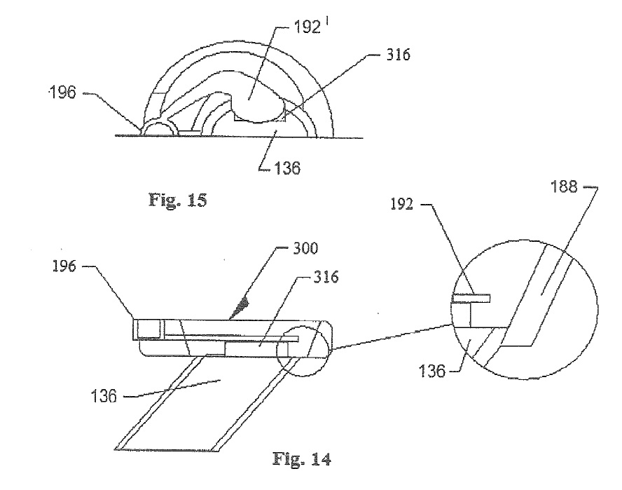

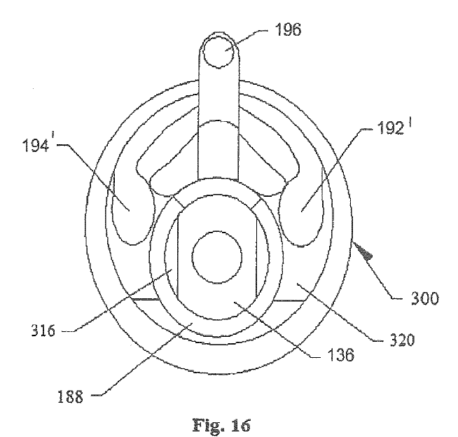

As the catheter is attached to the implant, the post 184 interfaces with the receiver 196 and as a consequence the surfaces 277 and 306 become aligned. Turning the control dial, e.g., clockwise, translates rod 152 forward, as well as the post and receiver, which in turn moves the locking tabs 192 and 194 (see FIGS. 14 and 15) radially outward, but still within the perimeter of the implant, unlocking the stem seal.

At the same time, the locking rod 152 causes the rocker arm to hingedly rotate under the implant sealing surface 285, contacting surface 282 to surface 285, forcing the surface 277 onto surface 306. The locking rod 152 causes rotation of the rocker arm, in one implementation, as described above, by using a ramp 185 on the locking rod to force rotation of a hinge coupled to the rocker arm.

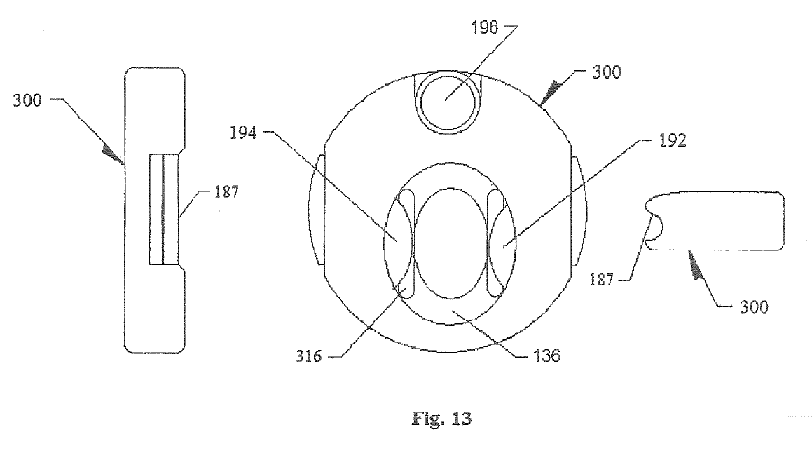

FIG. 13 shows locking edges 187 for use in the implant. In particular, the figure shows a top view of the implant surface indicating locking edges 187 which interlock with rocker arms on the catheter for a compression lock. FIGS. 14 and 15 show various views of the implant surface showing a sealing void, with a stem seal installed. Stem seal locking tabs are shown in position on the compression ridge.

As opposed to the interface locking mechanism embodiment of FIGS. 10-16, FIGS. 17-19 illustrate a different embodiment of an interface locking mechanism, this employing a dual cam sliding mechanism that removes compression of the stem seal but also attaches to the catheter housing.

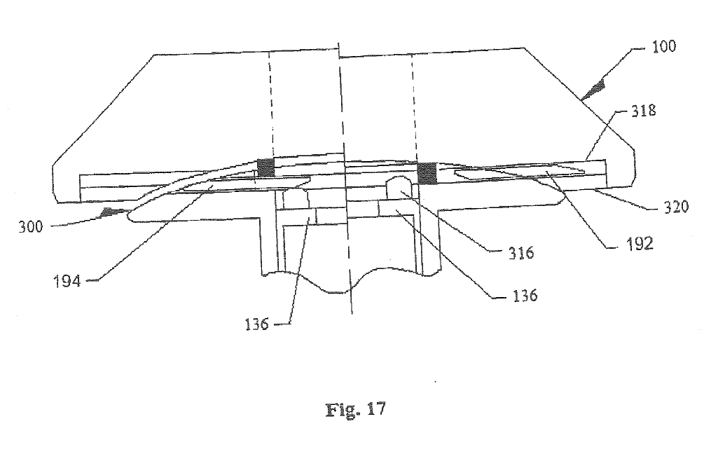

An embodiment of a set of locking tabs (192 and 194) are shown in FIGS. 17-19. By movement of the locking rod, a locking rod post 184 becomes disposed in the locking tab hole 196 and is forced in a distal direction. Of course, in an alternative embodiment, the same may be pulled in a proximal direction. The locking tabs 192 and 194 ride up respective surfaces and become engaged in corresponding tab slots in the catheter. A ramp aspect forces the two surfaces together, which then elastically compress the sealing surface 188. Simultaneously, the locking tabs 192 and 194 are forced apart, exposing the stem seal resident in the implant, and allowing the same to be removed so that dialysis may occur.

Compression ridges 316 are employed which are compressed by the locking tabs for the purpose of sealing the stem inside the passageway of the main implant. The stem seal door has two ribs that compress the plug in the passage. After plug insertion, the taper on both the passage and the plug creates the seal.

Elements 318 and 320 form two sides into which the locking tabs become disposed. Between elements 318 and 320 is a vertical dimension change forming a ramp.

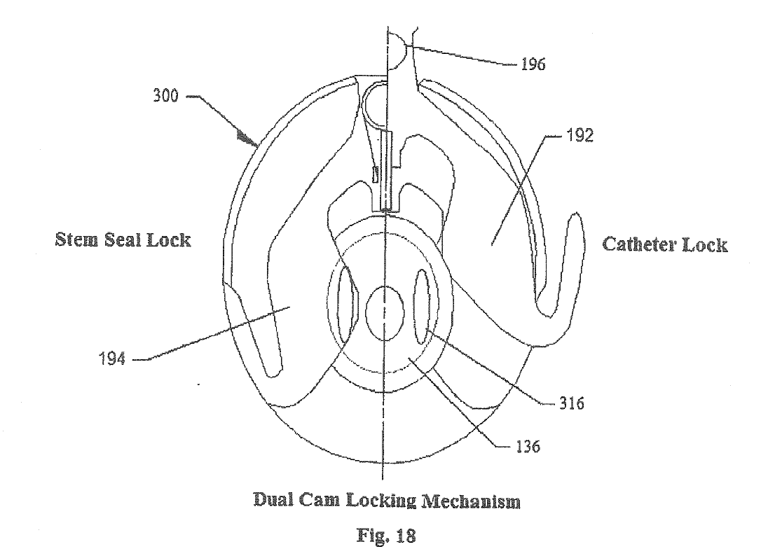

Referring to FIGS. 17 and 18, unlocked (left half) and locked (right half) configurations of the catheter and implant are shown, as well as the locking tabs 192 and 194. As FIG. 18 shows, the locking tabs, e.g., locking tab 194, may have an areal extent fully within the perimeter of the seal. On the other hand, when the locking rod post is forced forward, a shape of the surface (a cam, bump, or other elevation change) on which the locking tabs ride forces the locking tabs, e.g., locking tab 192, outward and upward. The same engages a corresponding locking tab slot in the catheter element. The initial entry may be flat, but a ramp feature may then be present which forces the catheter and the implant together at the seal area, compressing the sealing surface and making a seal.

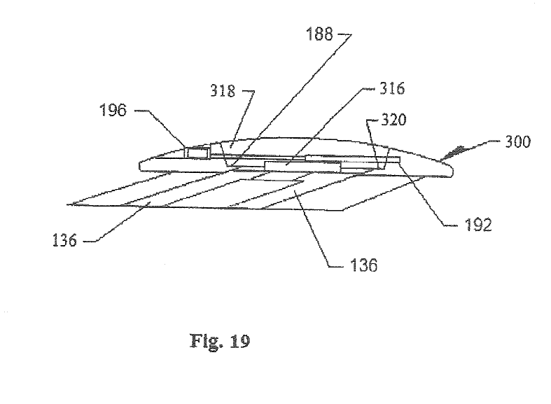

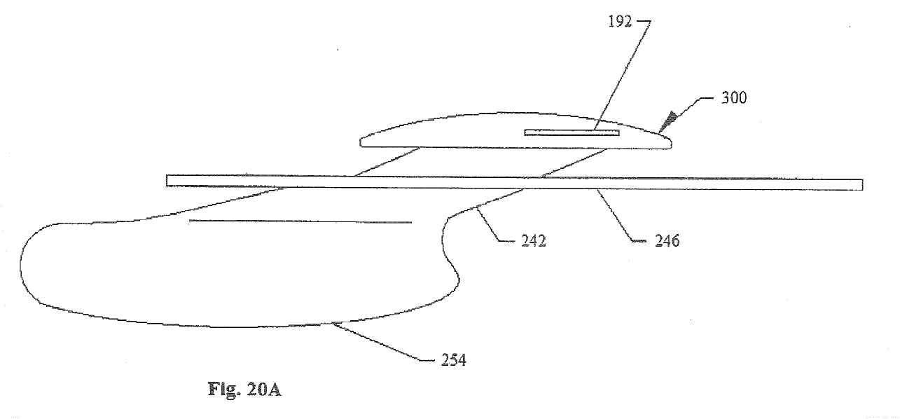

FIGS. 17 and 18 also show how, in general use, the locking tabs are positioned to prevent egress of the stem seal out of the implant. FIG. 19 shows a cutaway perspective view.

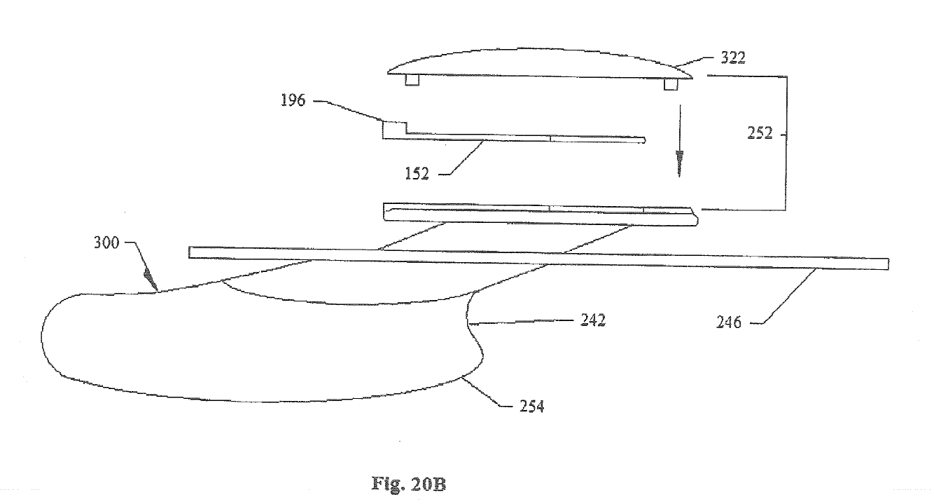

FIG. 20(A) shows a side view of the implant, including a protective seal 322. The locking tabs 192 and 194 may be connected in various ways, such as by a hinge, a spring, a torsional spring, or via other devices, so long as the above functions can be performed. They may also be formed of a unitary component, such as is a C-ring. FIG. 20(B) shows an exploded view of the embodiment of FIG. 20(A).

FIG. 21 shows a schematic of the extracorporeal blood circuit to and from the catheter housing, including an arterial supply tube 106, a venous return tube 108, a blood pump 112, a dialyzer 114, and a blood catheter cylinder 116. The housing of the catheter 100 is shown as housing 118. Blood is pumped out of the patient's blood vessel via the action of the blood pump 112, which is typically a roller pump but may be any suitable pump, on the arterial supply tube (following a suitable priming sequence), and the same is pumped through the dialyzer and then back into the patient's blood vessel via the venous return.

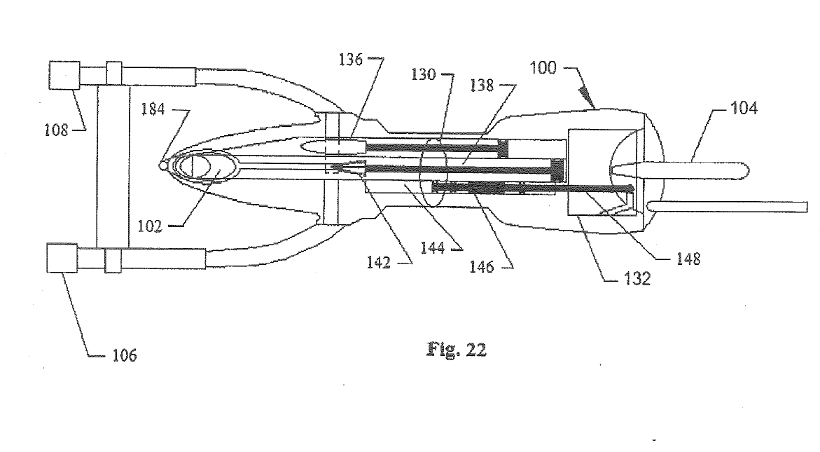

FIG. 22 shows a top schematic view of the catheter device according to an embodiment of the invention. This view shows the tube bundle arrangement 130 with a driving cam 132 and a control dial 104. Other components are also shown as are described elsewhere.

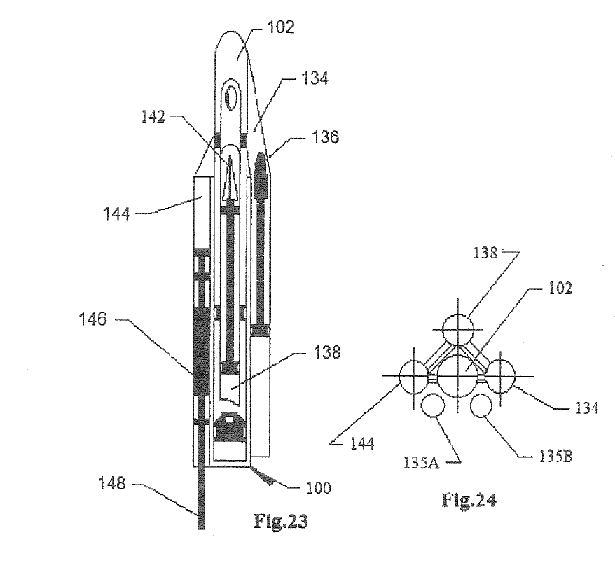

FIG. 23 shows a more detailed view of the tube bundle assembly, showing in particular the blood catheter 102 surrounded by a stem seal infusion lumen 134, a stem seal retraction lumen 138, and a spool lumen 144. The stem seal infusion lumen 134 includes a stem seal 136; the stem seal retraction lumen 138 includes a stem seal retraction device 142 (in the figure having retracted a stem seal), and the spool lumen 144 includes a spool 146 having a position controlled by spool rod 148.

FIG. 24 shows an end-on view of the tube bundle assembly of FIG. 23, showing blood channels 135a and 135b.

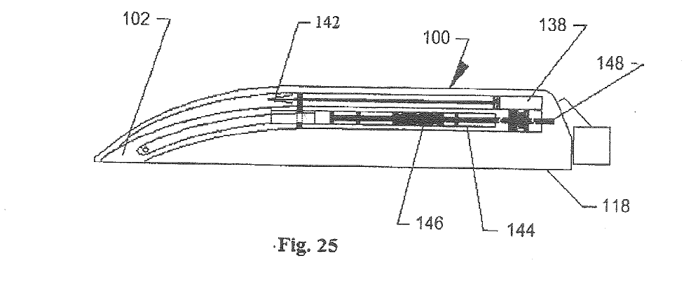

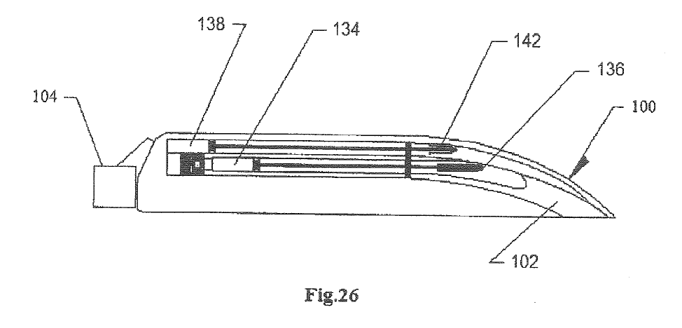

FIG. 25 shows a left-side view of the catheter device 100, and FIG. 26 shows a right-side view. These view illustrate, among other features, how the retraction and infusion (insertion) cylinders merge into the main blood catheter 102.

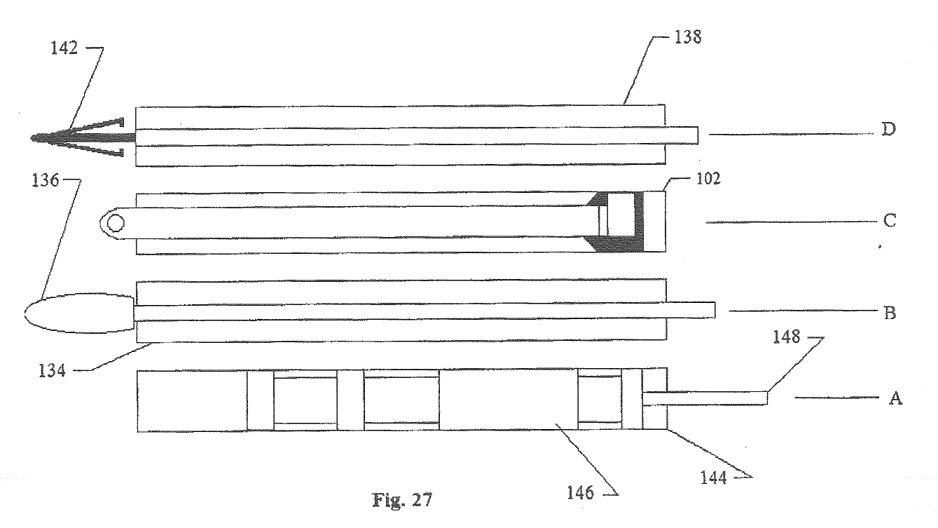

Each cylinder or lumen in the tube bundle assembly responds to pressurized saline and creates a mode for dialysis. In one implementation, rotation of a control dial diverts saline through a spool, and this ports the saline to the other cylinders or lumens. FIG. 27 shows a schematic view of the tube bundle cylinders, showing: (A) a spool cylinder or lumen 144 for diverting fluid to other lumens or cylinders for the separate modes; (B) a stem seal insertion lumen or cylinder 134 to insert a new seal at the end of dialysis; (C) a main blood catheter lumen or cylinder 102 for vascular access; and (D) a stem seal retraction lumen or cylinder 138, for manual insertion and hydraulic retraction. Of course, one of skill in the art will recognize, given this teaching, that various other steps may be automated or alternatively made subject to manual operation.

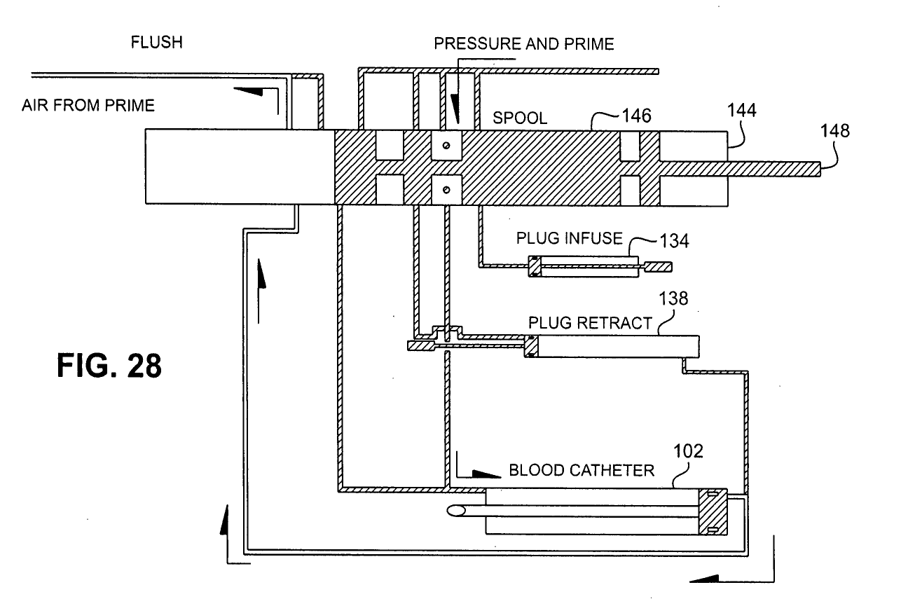

FIG. 28 shows a schematic view of the tube bundle, in the priming mode. In this mode, the blood catheter is retracted up inside a protective cap 168 (see FIG. 4). The priming mode serves to remove air from inside the catheter.

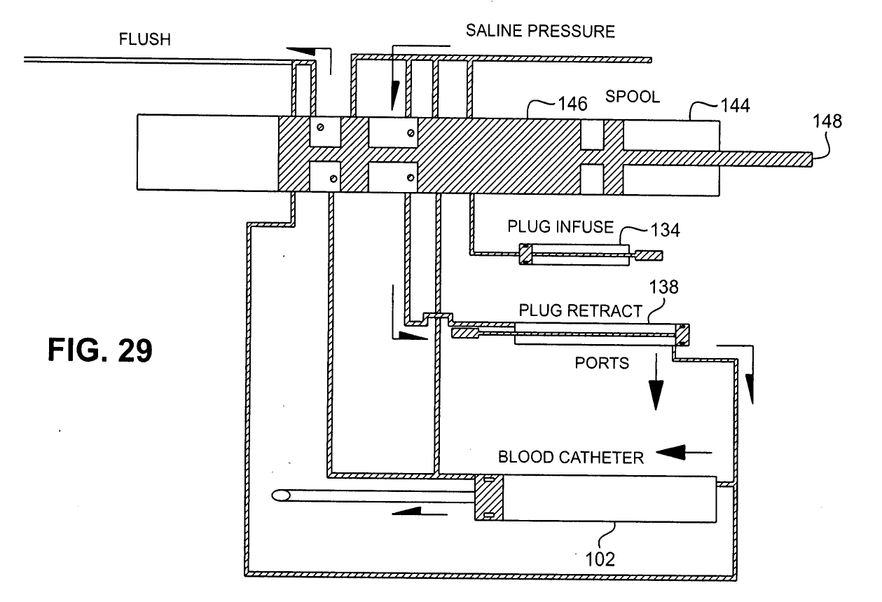

FIG. 29 shows a schematic view of the tube bundle 130, in the catheter infusion mode. Arrows are employed to indicate fluid direction. In the infusion mode, the stem seal 142 is retracted and the blood catheter 102 is infused. As shown by the spool position, the blood catheter 102 is primed with saline during this procedure, as is the stem seal (plug) retraction cylinder 138.

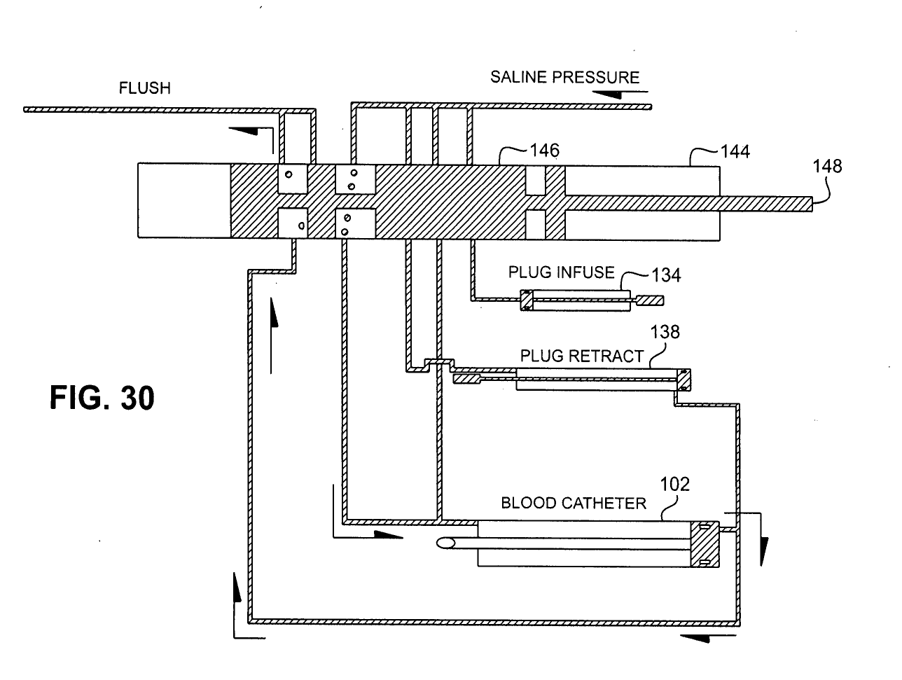

FIG. 30 shows a schematic view of the tube bundle 130, in the catheter retraction mode, whereby dialysis would be terminated. In this mode, the blood catheter 102 is retracted by a saline flush counter-current to that shown in FIG. 29. As shown by the spool position, pressurized saline is flushed into the blood catheter 102 from the left, forcing the piston to the right, and pulling (retracting) the blood catheter 102 out of the implant 300. The spool position also allows for flushing the forced pressurized saline out of the blood catheter 102.

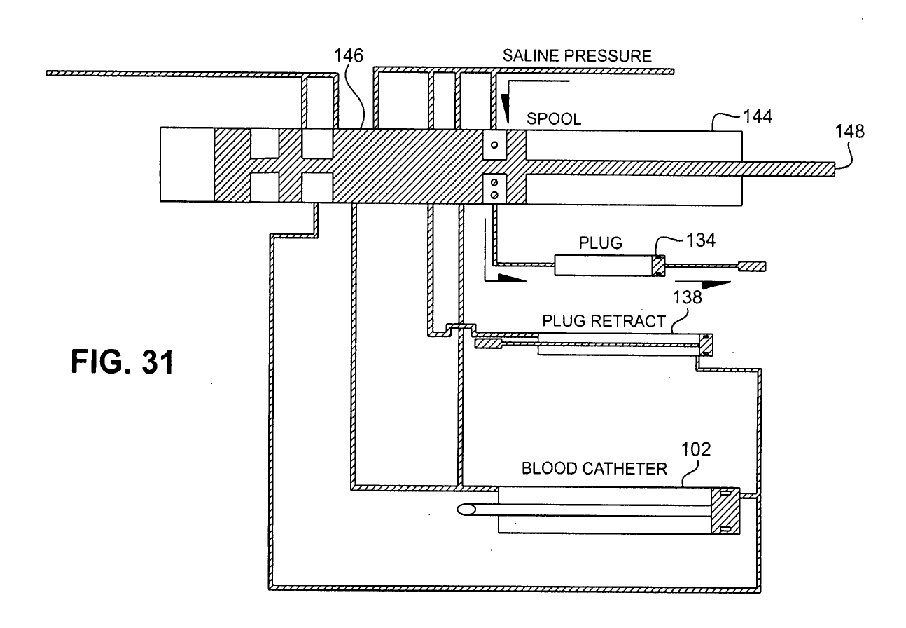

FIG. 31 shows a schematic view of the tube bundle 130, in the seal infusion mode. In this mode, a fresh stem seal is inserted into the implant following dialysis. As shown by the spool position, the stem seal infusion cylinder 134 is pressurized with saline, forcing the fresh stem seal into the implant.

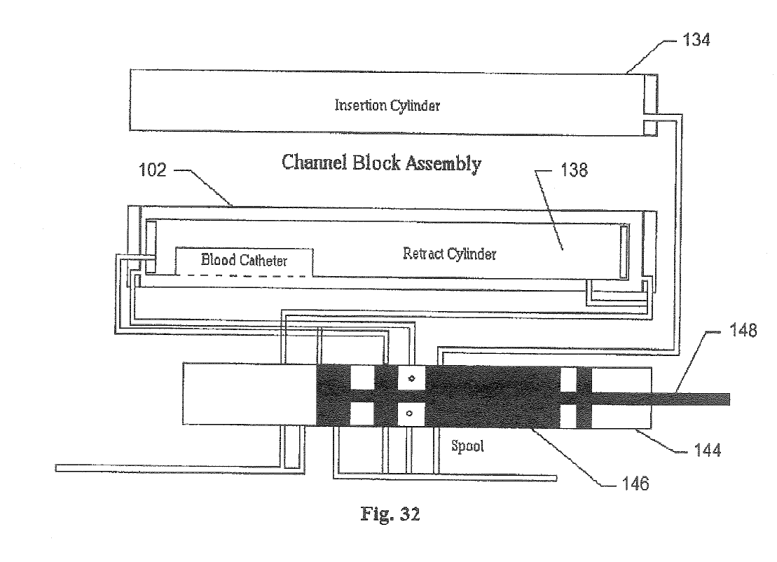

FIG. 32 shows a schematic view of the port configuration in the tube bundle assembly, showing in particular fluid pathways into and out of each cylinder.

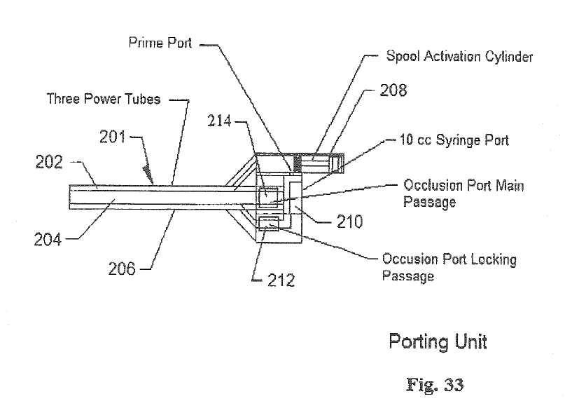

FIG. 33 shows a side sectional view of a porting unit which may be employed in the second embodiment of a catheter of the invention. In particular, a power tube bundle 201 is shown which is comprised of: tube 202 fluidically coupled to a spool activation cylinder 208; main passage tube 204 fluidically coupled to occlusion passage 214; and tube 206 fluidically coupled to occlusion passage 212. Syringe port 210 is also shown. The displacement of activation cylinder 208 is proportional to the displacement of the spool 146 and is used for visual reference for piston placement.

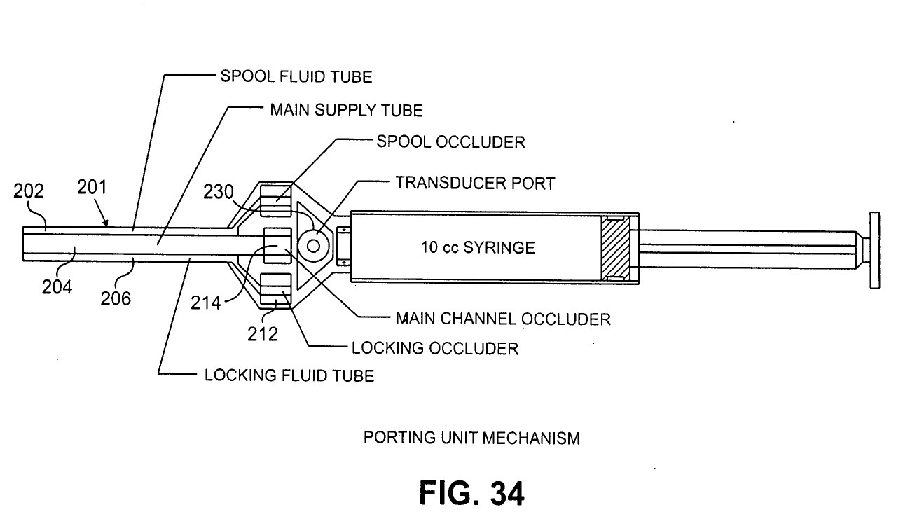

FIG. 34 shows a top view of a porting unit related to FIG. 33; however, here the porting unit is controlled by a pressure set point from a transducer 230 that couples transducer feedback unit. As the fluid pressurizes a cylinder in a mode, there is a maximum pressure that stops the infusion and either occludes that tube or alternatively waits for the next mode. Each mode has a calculated set point for pressure, and resistance to the piston reaction means that the travel is complete and that the mode is finished.

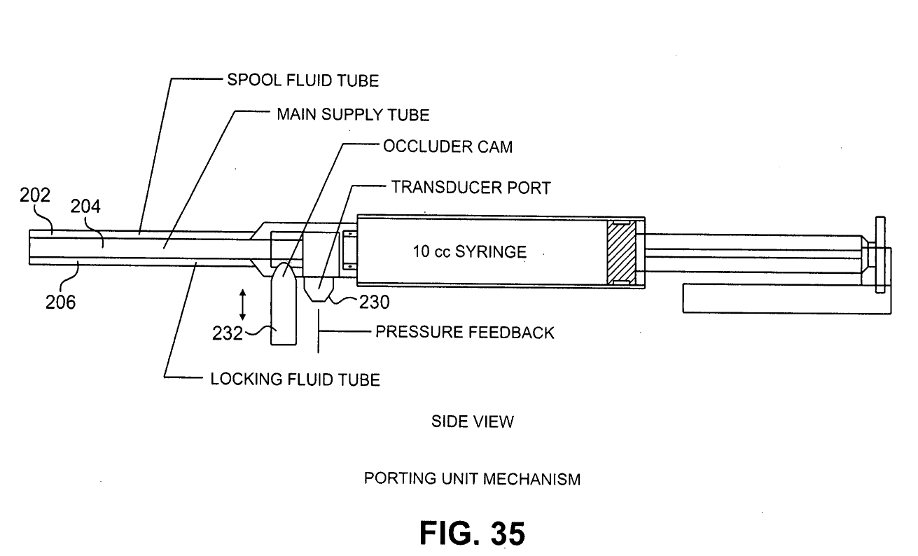

FIG. 35 shows a side view of this embodiment. This view also shows an occluder cam 232 that controls occlusion in a given tube 202, 204, or 206.

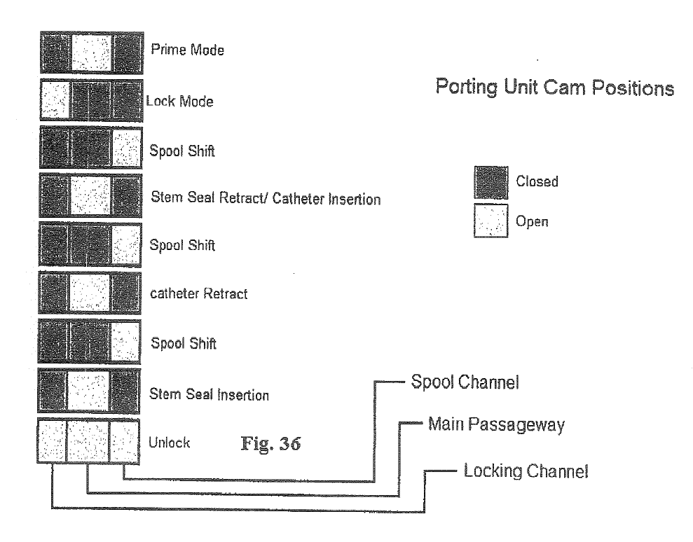

FIG. 36 illustrates a logic diagram for the porting unit, showing the appropriate occlusion positions for various modes. While this is displayed for the embodiment of cam-type occluders, any other type of occluder may also be employed. Indications are shown for the spool channel, the main passageway, and the locking channel.

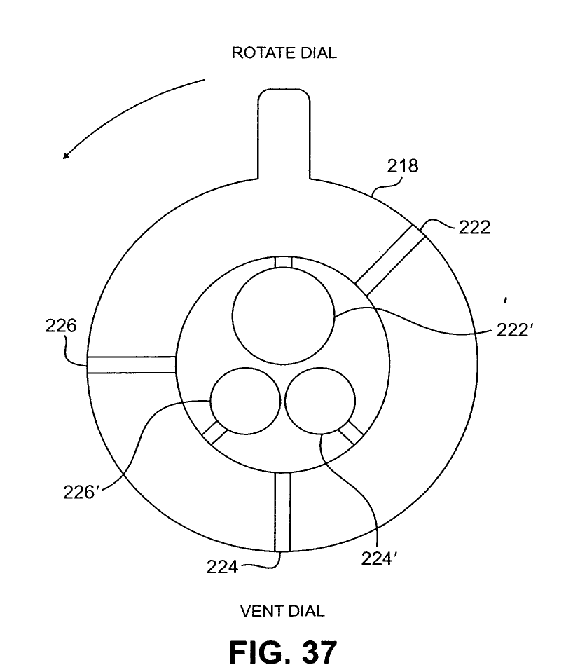

FIG. 37 shows a vent dial which may be employed with the second embodiment of the catheter. In this embodiment, aligning the fluid passages allows fluid movement. In more detail, in the automated system of the second embodiment, priming of the fluid lines and the porting mechanism is accomplished by opening the vent disk 218 on the back of the catheter, thereby venting out air from the three separate fluid lines via aligning passages 222, 224, and 226 with corresponding passages 222′, 224′, and 226′. Pushing the saline-filled syringe 216 forward through the porting unit chambers and towards the vent disk then primes all tubes. The vent disk is closed after fluid is seen to exit the ports.

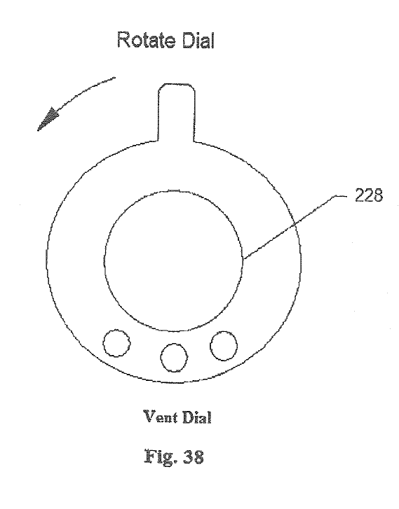

FIG. 38 shows another embodiment of a vent dial which may be employed with the second embodiment of the catheter. In this system, rather than aligning a set of aligning fluid passageways that are disposed on either side of a circle (see circle) concentric with the longitudinal axis of the dial, all the passageways are at substantially the same radius, and rotating the dial brings, e.g., three sets of passageways into alignment with three different passageways. In this way, all fluid passageways are disposed on a single side of the circle concentric with the longitudinal axis of the dial.

The porting unit supplies fluid pressure to the catheter device and forms part of the entire packaged assembly. In one implementation, a specialized pump may work in combination with the porting unit to allow for pressure infusion into three separate tubes that enter into the back of the catheter device, these allowing for pressure infusion to be diverted to the locking cylinder, the spool cylinder, and the main supply passage. One syringe unit is used in the porting unit with the attached porting unit, and the porting unit switches for fluid control from the syringe into the three catheter infusion lines. The fluid entering the catheter will divert according to the activation on the pump. The fluid in the lines may be in, for example, locked mode, pressure mode, or pulse infuse mode.

The locked mode sets the first infusion. In this mode, only the locking cylinder in the catheter receives fluid; the other two lines are closed, e.g., the main tube is occluded via occlusion passage 214. After full infusion, this line will be occluded using occlusion passage 212 in the porting unit for the full dialysis procedure. The next line to open is the main passage supply. This line stays open throughout the entire treatment and provides pressure for the cylinders in tube bundle operations. The third line is used to move the spool rod, e.g., and ⅛″ in a pulse method. Terminating the treatment is accomplished by powering down the hydro power unit and opening the porting unit ports, thus reversing the syringe enough to displace the fluid in the locking cylinder inside the catheter device.

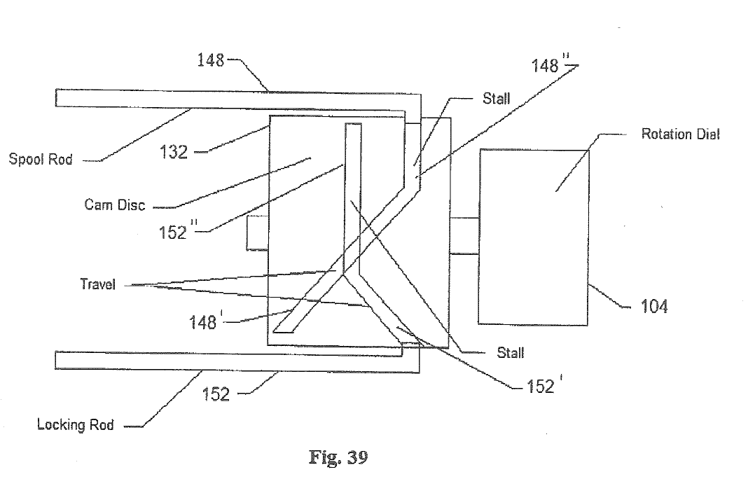

FIG. 39 shows a more detailed schematic view of a cam driving disk 132, in particular showing travel and stall modes, when rotating the control dial 104. The spool rod 148 has a travel channel 148′ and a stall channel 148″. The locking rod 152 has a travel channel 152′ and a stall channel 152″. The cam pushes the upper spool shaft in position for fluid porting in the tube bundle, and the lower locking shaft travels forward and back to lock or unlock the stem seal into the exposed surface of the implant.

The locking rod 152, as it moves forward, serves to lock the catheter to the implant in a manner described below. An occluder is employed once this lock is performed. By occluding the hydraulic pressure, the lock can be maintained without have to continue pump operation during the entire dialysis procedure.

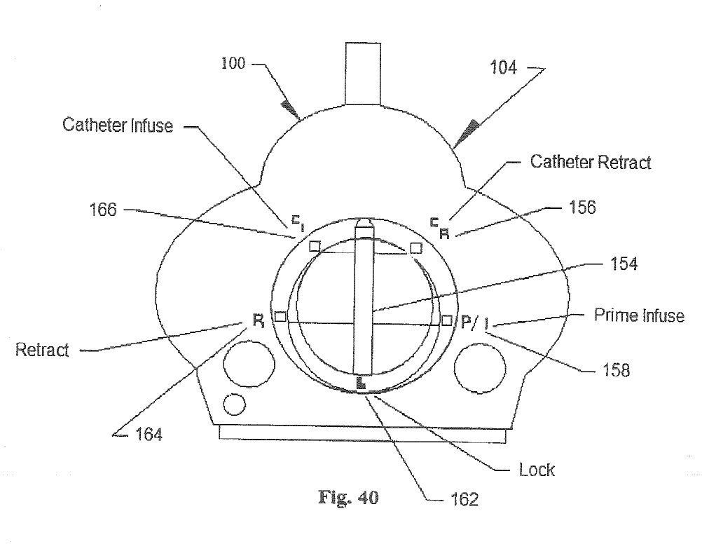

FIG. 40 shows a view of the rotation dial 104, which is disposed on one end of the catheter device. The dial 104 has a knob 154 which allows the system to be placed in the following configurations: a catheter retract position 156, a prime/infuse position 158, a lock position 162, a retract position 164, and a catheter infuse position 166. It is noted in this connection that in one embodiment the spool moves 1/16″ each time the dial is rotated one position.

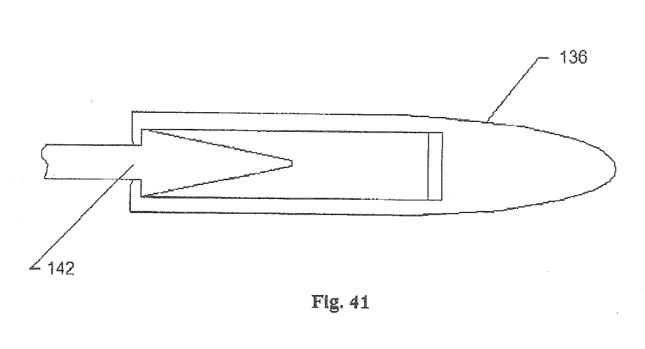

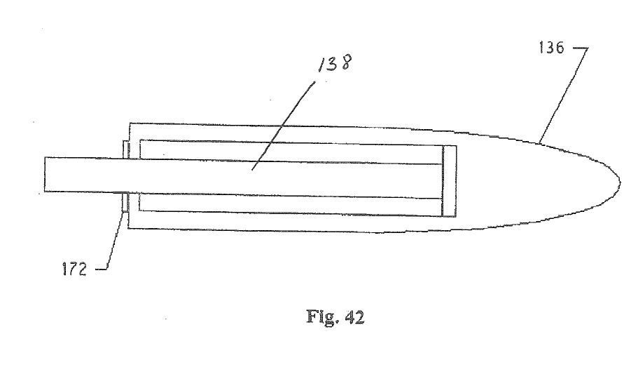

FIG. 41 shows a view of the stem seal retraction, showing in particular a stem seal rod 142 with a device such as a spear tip for attachment and removal of a previously-installed stem seal 136. FIG. 42 shows a view of the stem seal insertion, showing in particular a stem seal insertion device 172, where in this embodiment the stem seal insertion device 172 includes a push rod for seating in the implant 300. The push rod retracts following the locking of locking tabs positioned above the stem seal. In this way, a compression lock is achieved.

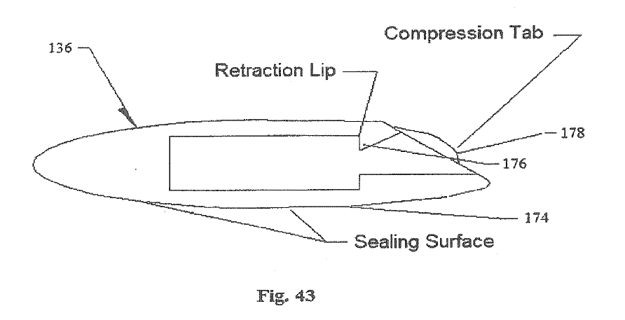

FIG. 43 shows a cutaway view of the stem seal 136. The stem seal 136 is shown with a broken perimeter to indicate that the length of the same may vary. The sealing surface 174 provides the primary frictional surface where locking occurs. The retraction lip 176 allows the stem seal retraction device to lock onto the stem seal for removal. The internal void allows compression of the components. At least one compression tab 178 may be provided, which is a volume of increased rigidity or stiffness, and which allows for enhanced compression and sealing.

### Operations

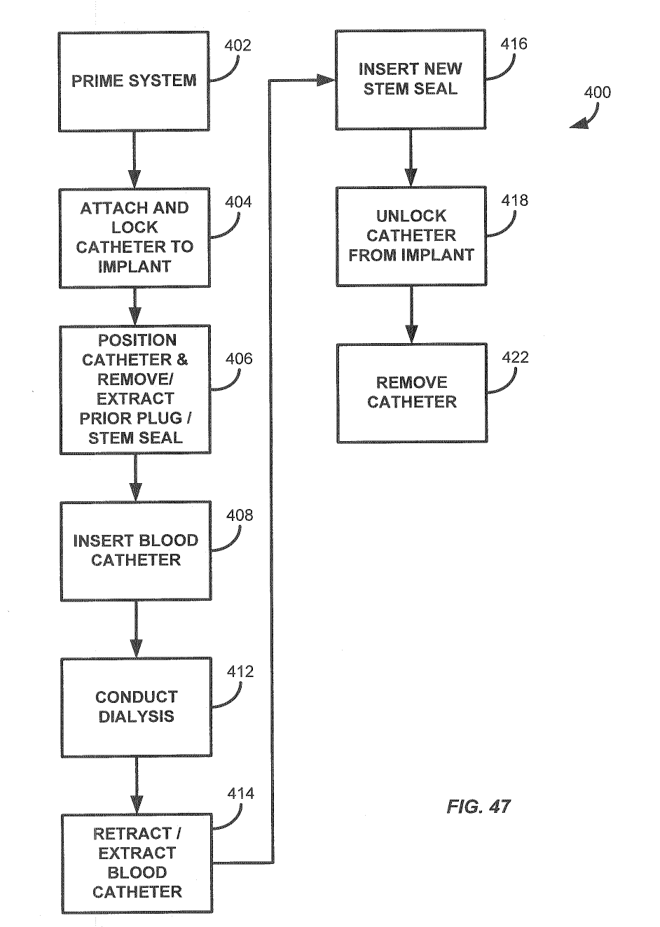

Referring to FIG. 47, as well as to the figures throughout, the mechanical functions and the hydraulic pathways in the catheter housing, and the steps involved for the dialysis procedure, will now be described. Generally, this description explains what the nursing staff controls and what the catheter may perform automatically or semi-automatically.

The following series of steps describe steps of operation where a hydro power unit is not employed.
Step 1. Prime (step 402): A syringe is filled with saline and loaded into the pressure unit (spring loaded), the primed line to the catheter port is connected, and the control knob is turned to ‘prime’. This retracts the catheter tube upward inside the housing; this is now the saline prime position for infusion. The distal tip of the catheter is now inside the catheter housing, and is still protected by the protective cap.
Step 2. Lock (step 404): The protective cap is removed from the catheter housing, and the tip of the catheter is placed on the implant's exposed surface, aligning the two surfaces respectively. The control knob is turned to the lock position, this turns the cam which moves the locking bar forward to press the locking tabs downward onto the implant surface, compressing both surfaces together. This is now the seal preventing air and blood leakage. As the tabs start to create compression, the same locking bar slides the seal door forward, retracting the cams inside the housing to expose the stem seal for extraction.
Step 3. Engage (step 406 (partial)): The upper tab on the catheter housing is pushed forward; this engages the distal tip of the spear into the implant's opening and into the plug seal. At this point the spear tip has locked onto the stem seal and is positioned for extraction.
Step 4. Stem Seal Retract (step 406 (partial)): The control knob is then rotated to the dialyze position, which starts an automated portion of the system. The pressurized saline is diverted through the spool and into the retraction cylinder, pushing the stem seal upward into the stowed position. The cylinder has now ported fluid out to the catheter retraction cylinder and into the catheter cylinder, inserting the blood catheter into the implant's passageway, and positioning the same parallel inside the vessel. This event also causes the prime flush to displace induced air.

The blood catheter may then be inserted for dialysis (step 408). Dialysis is then conducted (step 412). A typical dialysis dwell time is approximately 4 hours.
Step 5. Catheter Retract (step 414): The control knob is now rotated to the retraction position, which diverts fluid to the blood catheter cylinder and retracts the piston upward. As retraction of the blood catheter occurs, the previous fluid used for insertion is now used for the final flush of the implant's passageway. The passage is cleaned of residual blood and is ready for the new stem seal which is stowed in the catheter housing.
Step 6. Stem Seal Insertion (step 416): The control knob is now rotated to the stem seal insertion position, which channels fluid into the insertion cylinder, the same having a stowed stem seal. This is the final fluid port to complete the dialysis procedure.
Step 7. Unlock (step 418): The control knob is now turned back to its original position, which closes the seal door and reverses the locking tabs for removal.

The catheter may then be removed (step 422).

### Module Dialyzer

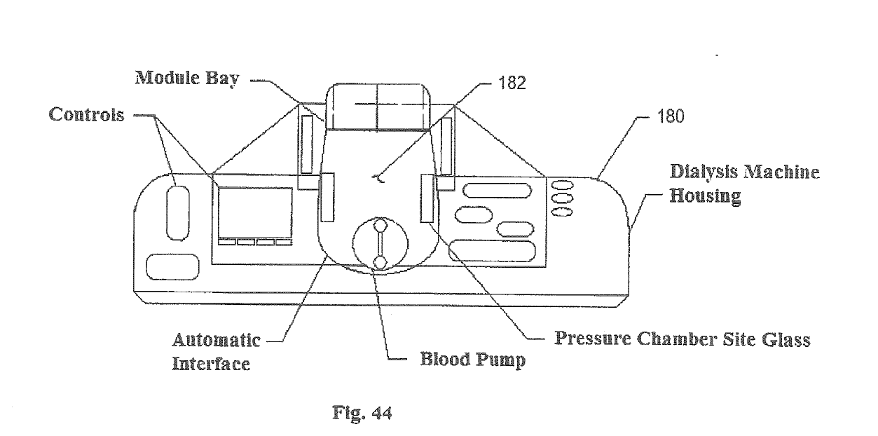

FIGS. 44-46 show a home module dialyzer 180. The home system 180 works on the idea of speed, safety, and convenience for the home user, easy loading of a module dialyzer 182 for the blood circuit, and the automation of a blood access device. The small table top machine accepts the module and attaches to its ports for fluid and pressure monitoring.

FIG. 44 shows an end schematic view of a home dialysis machine 180, with a module dialyzer installed.

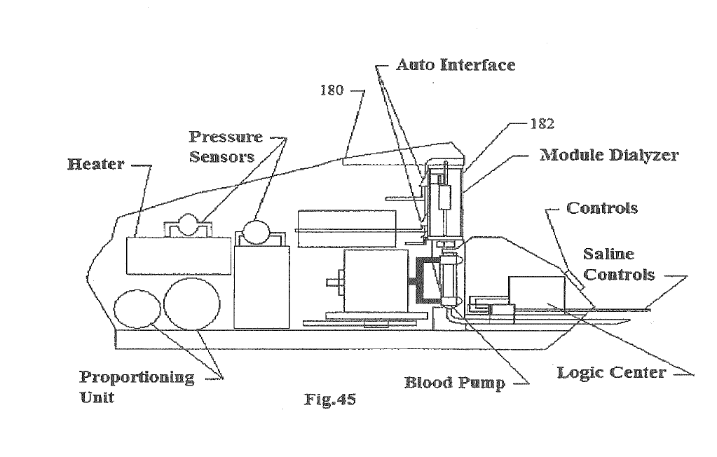

FIG. 45 shows a side sectional view of the home dialysis machine.

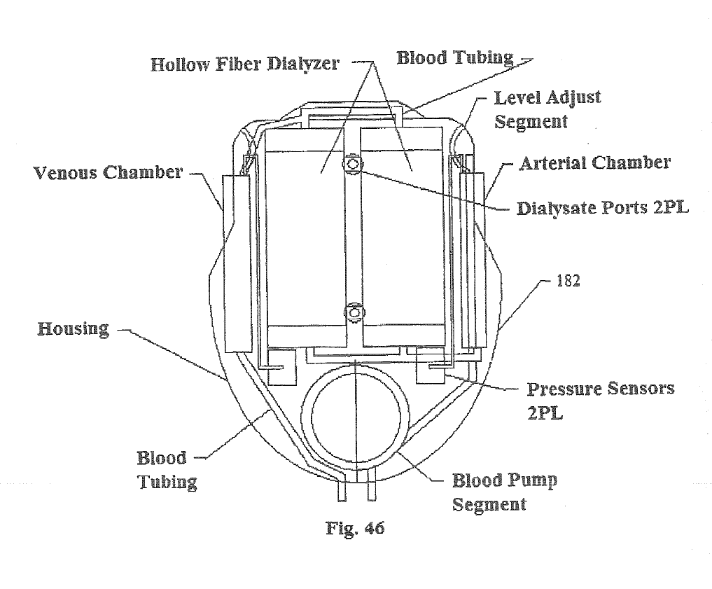

FIG. 46 shows a top sectional view of the module dialyzer.

The dialysis machine may have a compartment that houses a hydro-cylinder for the activation of the catheter. In such implementations, this may serve to pressurize the saline for catheter activation and provides constant pressure for the entire procedure.

This self-contained system provides clean and safe operation, as well as low operator error, for the ease of the patient, enabling the patient to initiate dialysis quickly and in a worry-free way. In this way, stresses are reduced from needle placement, connections, priming, set-up and take-down time, and post-clotting problems.

The module dialyzer 182 has the catheter and pressure cylinder as one unit. This configuration package enables the patient to start dialysis in less time, e.g., for three times a week or for daily dialysis.

The blood pump loads forward into the module's pumping segment, and the conical tipped rollers rotate as placement begins onto the blood tubing, allowing for travel on the segment for proper pumping.

The pumping unit may be disposed at the base of the module dialyzer, so that pumps may be inserted into and out of the unit in a convenient fashion. In this way, more space and volume is available for dialyzer surface area, allowing for a smaller and more convenient overall dialyzer.

This module concept enables the machine to do most of the interface by automatic connections. Blood pumping, arterial and venous pressures, dialysate, and heparin are ported after the module has been loaded in. After loading, air is removed in both blood and dialysate compartments in the upward direction in a concurrent flow. After the priming is complete, dialysate flow is reversed to create countercurrent flow for proper dialysis.

Numerous advantages inure to certain embodiments of the invention. It was noted above in connection with FIG. 2 that the dial may be oriented at a variety of angles to allow for ease of user operation.

The embodiments of the invention above are designed to help lower the infection rate and lessen the problems associated with the dialysis routine, in the clinical facility or in a home care setting. The catheter has the ability in some embodiments to self-lock, load, seal, and prime without risk of contamination. Various automation functions enable the procedure to have less hands-on involvement.

The implant further assists the catheter's interface ability to create a hands-free process to connect the two devices together for blood access. Because the low profile and the stable structure may be protected by a three-way infection topical barrier seal, infection to the implant is minimal. Smooth internal lumen designs and external surface interfaces make for a convenient biocompatible interface between body and foreign body.

While the system has been described with respect to certain embodiments, it is clear that the scope of the invention is broader than the described embodiments. For example, the system may be employed to allow vascular access for any purpose besides dialysis. The system may be employed to ease the introduction of microcatheters into the vascular system. The system need not require a user-rotatable dial: rather, the dial may be rotated by a motor or other automated system, according to a predetermined programmed scheme. The tube bundle may be operated by micromotors instead of hydraulics, or by any other sort of system or device which can insert and retract components. The tube bundle may even be operated manually, such as by a technician or physician; that is, the distal end of the tube bundle may be open and may allow a technician to retract a pre-inserted stem or plug, insert a blood catheter, conduct dialysis, retract a blood catheter, and replace a stem seal, all by manually inserting such components in a multi-lumen tube bundle with an open end. Besides the ways described above for connecting the catheter to the implant, numerous other variations will be apparent to one of ordinary skill in the art given this teaching. While the system has been described with respect to certain spacings of inlets and outlet for the blood flow to a dialysis machine, the inlets and outlets may be arranged in a number of other ways as well. In lieu of the stem seal, various valving arrangements may be employed instead. The blood catheter need not fully enter the area of the implant; in fact, in some implementations, the blood catheter may remain in the catheter area—in these cases, once the catheter is connected to the implant and the stem seal removed, blood may flow directly to a catheter-resident blood catheter (which in this case may be merely a lumen within the catheter housing).

Accordingly, the scope of the invention is to be limited only by the claims appended hereto.

What is claimed is:

1. A method of conducting a dialysis procedure using a semi-automated process in which an implanted graft is accessed by a catheter, comprising:
a. attaching a catheter to an implant;
b. opening a blood passageway between the catheter and the implant;
c. conducting a dialysis procedure;
d. closing a blood passageway between the catheter and the implant
e. removing the catheter from the implant.

2. The method of claim 1, further comprising priming the catheter.

3. The method of claim 1, wherein the attaching includes:
a. contacting a distal end of the catheter to an exposed surface of the implant;
b. engaging one or more rocker arms with an upper surface of the exposed surface, such that rotation of the rocker arms forces the distal end of the catheter against the exposed surface of the implant.

4. The method of claim 3, wherein the engaging is caused by translating a locking rod forward, a ramp on the locking rod forcing upward movement of an element coupled to the rocker arm.

5. The method of claim 1, wherein the attaching includes:
a. contacting a distal end of the catheter to an exposed surface of the implant;
b. forcing a locking tab extending from the implant into a ramped slot on the catheter, such that entry of the locking tab into the ramped slot forces the distal end of the catheter against the exposed surface of the implant.

6. The method of claim 5, wherein the forcing is caused by translating a locking rod forward, the locking rod forcing movement of the locking tabs against a cam, the cam forcing movement of the locking tabs upward and outward into movement of an element coupled to the rocker arm.

7. The method of claim 1, wherein the opening includes removing a stem seal from the implant.

8. The method of claim 7, wherein the removing a stem seal from the implants includes extending a spear at least partially through the catheter in a distal direction to contact and engage the stem seal, and pulling the spear in a proximal direction to remove the stem seal.

9. The method of claim 1, further comprising extending a blood catheter through the catheter to fluidically engage the blood flow in the graft.

10. The method of claim 1, wherein the closing includes inserting a stem seal at least partially through the catheter and into the implant to seal the implant against egress of blood.

11. The method of claim 1, wherein the attaching, opening, and closing are performed using hydraulics.

12. The method of claim 1, wherein the attaching, opening, and closing are performed using micromotors.

13. The method of claim 1, wherein a user selection of opening, conducting, or closing is performed using user rotation of a control dial.

14. The method of claim 1, wherein a user selection of opening, conducting, or closing is performed using a controllable hydraulic pump.

15. A catheter system for performing dialysis, the catheter system for connecting to an implant, the implant saddling a blood vessel graft, the system comprising:
a. means for attaching a catheter to an implant;
b. means for opening a blood passageway between the catheter and the implant;
c. means for conducting a dialysis procedure;
d. means for closing a blood passageway between the catheter and the implant.

16. The system of claim 15, wherein the means for attaching the catheter to an implant includes a set of rocker arms that extend from the catheter and engage a lip on the implant.

17. The system of claim 15, wherein the means for attaching the catheter to an implant includes a set of locking tabs that extend from the implant and enter a ramped slot on the catheter.

18. The system of claim 15, wherein the means for opening a blood passageway include means for removing a stem seal from the implant.

19. The system of claim 15, wherein the means for closing a blood passageway include means for inserting a stem seal into the implant.

20. An implant for performing dialysis, the implant saddling a blood vessel graft and allowing connection to a catheter, the implant comprising:
a. a saddle system for coupling to a blood vessel graft;
b. means for attaching the implant to a catheter; and
c. a seal to prevent blood egress from an interior of the implant.

21. The implant of claim 20, wherein the seal is a stem seal or plug.

22. The implant of claim 20, wherein the means for attaching the implant to a catheter is a set of locking tabs coupled to a receiver, such that translation of the receiver moves the locking tabs against a cam, wherein the locking tabs are forced in a direction towards a slot in the catheter.

23. The implant of claim 20, wherein the means for attaching the implant to a catheter is a surface, a rim, or a lip, which may be engaged by a rocker arm on the catheter.
